SEQUENCE LISTING (8 sequences)

SEQ ID NO: 1 - RNA, 19 nt, Artificial Sequence
  siRNA
acaagaaaguagaggguau

SEQ ID NO: 2 - RNA, 19 nt, Artificial Sequence
  siRNA
acaccuaguuucagagaau

SEQ ID NO: 3 - RNA, 19 nt, Artificial Sequence
  siRNA
ccucagaauaauuguguga

SEQ ID NO: 4 - RNA, 19 nt, Artificial Sequence
  siRNA
cagcaaagguacuugauac

SEQ ID NO: 5 - RNA, 19 nt, Artificial Sequence
  Control siRNA
ugguuuacauguuguguga

SEQ ID NO: 6 - RNA, 19 nt, Artificial Sequence
  Control siRNA
ugguuuacauguuuuccua

SEQ ID NO: 7 - DNA, 20 bp, Artificial Sequence
  Primer
gtgaggctgcagtgattgtg

SEQ ID NO: 8 - DNA, 20 bp, Artificial Sequence
  Primer
gattggtgagcgtgccgtat

(19) United States
(12) Patent Application Publication
(10) Pub. No.: US 2024/0110155 A1
(43) Pub. Date: Apr. 4, 2024

(54) A DUAL CARDIAC-BLOOD MODEL SYSTEM FOR DISEASE MODELLING AND DRUG SCREENING

(71) Applicant: GENOME BIOLOGICS UG, Kronberg im Taunus (DE)

(72) Inventors: Jaya KRISHNAN, Kronberg im Taunus (DE); Duc Minh PHAM, Offenbach (DE)

(73) Assignee: GENOME BIOLOGICS UG, Kronberg im Taunus (DE)

(21) Appl. No.: 18/257,884

(22) PCT Filed: Dec. 17, 2021
(86) PCT No.: PCT/EP2021/086634

§ 371 Date: Jun. 16, 2023

(30) Foreign Application Priority Data
Dec. 17, 2020 (EP) 20214892.0

(51) Int. Cl. C12N 5/077

(52) U.S. Cl. CPC C12N 5/0657; C12N 2500/02; C12N 2502/11; C12N 2503/02; C12N 2503/04

(57) ABSTRACT

The present invention relates to a cardiac cell culture model system composed of a cardiac organoid and blood or blood precursor cells. The cardiac cell culture model system serves as a model for the heart, and the present invention also relates to drug screening with the cardiac cell culture model system investigating the impact of the drug on the heart.

The present invention relates to a cardiac cell culture model system composed of a cardiac organoid and blood or blood precursor cells. The cardiac cell culture model system serves as a model for the heart, and the present invention also relates to drug screening with the cardiac cell culture model system investigating the impact of the drug on the heart.

### BACKGROUND OF THE INVENTION

CHIP (Clonal Hematopoiesis of Indeterminate Potential) results from acquired gene mutations in myeloid and lymphoid cells in absence of detectable hematologic malignancy.

The CHIP clonal subpopulations infiltrate the heart and lead to inflammation of the cardiac tissue and to cardiomyocyte death and consequent heart failure.

In addition, CHIP is a risk factor for other CVD development, such as coronary heart disease and early onset myocardial infarction.

No therapy is available, creating high unmet need for selective targeting of the CHIP subpopulations.

To establish a drug testing platform, crucial elements are needed, i.e. a 2-component system (both cardiac tissue and blood or blood precursor cells) and a mature cardiac tissue mimetic.

Based on the above-mentioned state of the art, the objective of the present invention is to provide means and methods to provide a cardiac cell culture model system for disease modelling and drug screening. This objective is attained by the subject-matter of the independent claims of the present specification, with further advantageous embodiments described in the dependent claims, examples, figures and general description of this specification.

### SUMMARY OF THE INVENTION

The invention relates to the culturing of cardiac organoids with myeloid, lymphoid, or hematopoietic cells, such that it enables the biological interaction of these two different entities. This maintained co-culture enables the monitoring and assessment of the infiltration of the myeloid/lymphoid/hematopoietic stem cells into the organoid, as well as other interactions between the organoids and the myeloid/lymphoid/hematopoietic stem cells, and of phenotypes resulting from the interactions. When using genetically modified myeloid/lymphoid/hematopoietic stem cells, one can evaluate the consequences that those genetic modifications have on the myeloid/lymphoid/hematopoietic stem cells' interactions with the cardiac organoids. Additionally, when using genetically edited, diseased, or otherwise modified cardiac organoids, one can evaluate the consequences of those features on the myeloid/lymphoid/hematopoietic stem cells' interaction with the cardiac organoids.

At a first complexity level, the combination of the two entities (myeloid/lymphoid/hematopoietic stem cells) allows for simultaneous communication between them via factors and vesicles secreted in their surroundings and in the media. This allows for assessment of the effects of this communication that would not be possible without the sustained maintenance of both.

As a second complexity level, the infiltration of the cells into the cardiac tissue means that myeloid/lymphoid/hematopoietic stem cells enter the cardiac tissue and come in close proximity to the cardiac cells. This infiltration allows for the interaction between the two entities (i.e. cardiac tissue and invading cells), not only to secreted factors and vesicles, but also includes interaction through surface proteins, mechanical interactions, extracellular matrix modifications that indirectly affect neighbouring cell types and other interactions. In this way, the effects of the CHIP cells on the cardiac tissue can be assessed in a manner and complexity that is not possible without the combination and close contact of the different entities.

Genetic mutations that cause CHIP can occur at cells in different differentiation stages, from hematopoietic stem cells all the way to terminally differentiated progeny cell types. In addition, mutations may occur at an earlier stage but have a phenotypic effect only in the further differentiated cell types. Lastly, some mutations may occur during earlier stages and, while carried by all progeny, only have a phenotypic effect in some of the derived cell types.

As a result, the invention includes the use of myeloid/lymphoid/hematopoietic stem cells at various differentiation stages in combination with the cardiac organoid, with the option to affect and drive differentiation directly in this dual system.

A first aspect of the invention relates to a method for pharmaceutical compound screening. The method comprises the steps
i. providing in cell culture/ex vivo
   a. a cardiac organoid comprising
      a plurality of cardiomyocytes;
ii. contacting said cardiac organoid with
   b. a plurality of myeloid cells, or
   a plurality of lymphoid cells, or
   a plurality of hematopoietic stem cells;
thereby yielding a cardiac cell culture model system,
iii. maintaining said cardiac cell culture model system under conditions of cell culture;
wherein a pharmaceutical compound of interest is contacted with
   the myeloid cells or the lymphoid cells or the hematopoietic stem cells, before they are contacted with the cardiac organoid; or
   the cardiac cell culture model system after step ii,
iv. detecting a read-out of the effect of said pharmaceutical compound on the cardiac cell culture model system,
v. optionally, repeating steps i-iv with a different pharmaceutical compound.

A second aspect of the invention relates to a cardiac cell culture model system as described in the first aspect and its embodiments.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The term mutation in the context of the present specification refers to a non-silent alteration in the nucleotide sequence of a coding region of a gene. Mutations include, but are not limited to insertions, deletions, and/or substitutions of nucleotides. Mutations include, but are not limited to loss-of-function mutations, gain-of-function mutations, and dominant-negative mutations.

The term CHIP mutation in the context of the present specification refers to a mutation with clonal hematopoiesis of indeterminate potential. A CHIP mutation is a mutation that happens in somatic cells (can be at any stage of cell differentiation) that leads to a clonal hematopoiesis phenotype. The phenotype is the development of a clonal population of myeloid or lymphoid cells but in the absence of detectable hematologic malignancy. The establishment of a clonal population may occur when a stem or progenitor cell acquires one or more somatic mutations that give it a competitive advantage in hematopoiesis over the stem/progenitor cells without these mutations. A variant allele frequency (VAF) of over 2% is one of the current thresholds for defining the mutation as a CHIP mutation, meaning that the mutation is found in 2% of the genetic material in the blood, and given that it happens in a single allele, this means that the mutation is observed in 4% of the cells (which have only 1 of 2 alleles mutated). Practically this means that any underlying mutation(s) which has enabled this clonal population to take over a big part of the blood cells will be a CHIP mutation. Thus, a CHIP mutation is a mutation that leads to an increased frequency of blood cells carrying this CHIP mutation inside an individual. Clonal hematopoiesis does not typically give rise to noticeable symptoms, but does lead to increased risk of cardiovascular disease. In one embodiment of the invention, the impact of CHIP mutations on the interaction of blood cells with the cardiovascular system is investigated.

The term genetic origin in the context of the present specification refers to the whole genome of a cell. If cells are of different genetic origin, then their genome differs in a least one base pair. In certain embodiments, cells are of different genetic origin if one of the cells carries at least one mutation and the other one does not. In certain embodiments, cells are of different genetic origin if they originate from different individuals.

The term cardiac organoid in the context of the present specification refers to an artificial three-dimensional structure comprising cells occurring in heart tissue, wherein the artificial three-dimensional structure mimics responses to physiological and/or biochemical and/or molecular reactions of native heart tissue.

### DETAILED DESCRIPTION OF THE INVENTION

A first aspect of the invention relates to a method for pharmaceutical compound screening. The method comprises the steps
i. providing in cell culture/ex vivo
   a. a cardiac organoid comprising
      a plurality of cardiomyocytes;
ii. contacting said cardiac organoid with
   b. a plurality of myeloid cells, or
   a plurality of lymphoid cells, or
   a plurality of hematopoietic stem cells;
thereby yielding a cardiac cell culture model system,
iii. maintaining the cardiac cell culture model system under conditions of cell culture;
wherein a pharmaceutical compound of interest is contacted with
   the myeloid cells or the lymphoid cells or the hematopoietic stem cells, before they are contacted with the cardiac organoid; or
   the cardiac cell culture model system after step ii,
iv. detecting a read-out of the effect of said pharmaceutical compound on the cardiac cell culture model system,
v. optionally, repeating steps i-iv with a different pharmaceutical compound.

In certain embodiments, the myeloid cells or the lymphoid cells or the hematopoietic stem cells are contacted with the pharmaceutical compound, before they are contacted with the cardiac organoid. In certain embodiments, the cardiac cell culture model system is contacted with the pharmaceutical compound after step ii.

In certain embodiments, step iii may allow for the myeloid or lymphoid cells or hematopoietic stem cells to invade the cardiac organoid depending on their cell-cell interaction. In certain embodiments, the cardiac cell culture model system is maintained under conditions of cell culture for 2 to 4 days. In certain embodiments, the cardiac cell culture model system is maintained under conditions of cell culture for 3 days. Conditions of cell culture are at 37° C., with 1% O₂, and with 5% CO₂.

In certain embodiments, the cardiac organoid is a cardiomyocyte monoculture comprising:
cardiomyocytes.

In certain embodiments, the cardiac organoid is a cardiomyocyte biculture comprising:
cardiomyocytes; and
fibroblasts.

In certain embodiments, the cardiac organoid is a triculture comprising:
cardiomyocytes;
endothelial cells; and
fibroblasts.

In certain embodiments, the cardiac organoid is a self-organized cardiac organoid comprising
epicardium;
myocardium;
endocardium; and
cardiac lumen,
wherein said self-organized cardiac organoid comprises:
   cardiomyocytes;
   endothelial cells;
   fibroblasts;
   smooth muscle cells;
   pericytes;
   neurons/sino-atrial node cells.

In certain embodiments, the myeloid cells or lymphoid cells or hematopoietic stem cells comprise a mutation.

In certain embodiments, the read-out is detected as cell viability of the cardiac organoid. In certain embodiments, the read-out is detected as cell viability of myeloid cells or lymphoid cells or hematopoietic stem cells. In certain embodiments, the read-out is detected as cytotoxicity of the lymphoid cells onto the cardiac organoid. In certain embodiments, the read-out is detected as cardiomyocyte cell death. In certain embodiments, the read-out is detected as proliferation of the cells of the cardiac organoid. In certain embodiments, the read-out is detected as proliferation of the myeloid cells or lymphoid cells or hematopoietic stem cells. In certain embodiments, the read-out is detected as proliferation of macrophages. In certain embodiments, the read-out is detected as contractility. In certain embodiments, the read-out is detected as mitochondrial activity. In certain embodiments, the read-out is detected as metabolism of the cells. In certain embodiments, the read-out is detected as cell infiltration of the myeloid or lymphoid or hematopoietic stem cells into organoid. In certain embodiments, the read-out is detected as inflammation (molecular profiling). In certain embodiments, the read-out is detected as reactive oxygen species quantification. In certain embodiments, the read-out is detected as reactive nitrogen species quantification. In certain embodiments, the read-out is detected as fibrosis of the cardiac organoid. In certain embodiments, the read-out is detected as genomics of the cells of the system. In certain embodiments, the read-out is detected as transcriptomics of the cells of the system. In certain embodiments, the read-out is detected as proteomics of the cells of the system. In certain embodiments, the read-out is detected as metabolomics of the cells of the system.

### Read-Outs

Cell viability is a measure of the proportion of live, healthy cells within a population. Cell viability assays are used to determine the overall health of cells, optimize culture or experimental conditions, and to measure cell survival following treatment with compounds, such as during a drug screen. Examples of assays include Alamar Blue, MTT Assay, hsTNT/cTNT Assay, Secreted Reporters, etc.

Cytotoxicity is the quality of being toxic to cells. It can be measured by detection of apoptotic and necrotic markers, lineage specific cell death reporters, indirectly by measuring decrease in viability and others.

Cardiomyocyte cell death;—this is the same as the cell viability with cardiac specific markers, like hsTNT/cTNT.

Proliferation assays monitor the number of cells over time, the number of cellular divisions, metabolic activity, or DNA synthesis. Examples include (as above plus EdU/Ki-67, Phospho-histone H3, Aurora B (late telophase), Secreted Reporters.

Cardiac contractility and contractile strength measurement involves the use of different compounds with known clinical effects that induce direct contractile changes of cardiomyocytes or cardiac tissues. An example method is IonOptix, beat counting, MEA System measurements, etc. but may involve any assay that measures the amplitude, contractile velocity, total signal under transient curve and other contractile parameters. (Brian D. Guth Front. Pharmacol., 9 Aug. 2019, doi.: 10.3389/fphar.2019.00884; https://www.axionbiosystems.com/applications/cardiac-activityfinotropy#example-data).

Mitochondrial activity assays measure the disruption of mitochondrial function, which can be detected using a variety of fluorescence-based assays including measurements of mitochondrial calcium, superoxide, mitochondrial permeability transition, and membrane potential (https://www.thermofisher.com/de/de/home/life-science/cell-analysis/cell-viability-and-regulation/apoptosis/mitochondria-function.html#MitoFCA).

Metabolism assays monitor the changes in metabolism, the balance between anabolic (including energy production) and catabolic processes (biomacromolecule synthesis). Since metabolic pathways have to rewire depending on cell needs and environment, these assays can reflect the cells' physiological health. Metabolic assays include, but are not limited to SeaHorse, Lactate Assay, JC10 Assay, etc. (https://www.promega.com/-/media/files/promega-worldwide/europe/promega-benelux/webinars-and-events/discoverglo2018/metabolism-jolanta-vidugiriene.pdf?la=en).

Cell infiltration of the myeloid or lymphoid or hematopoietic stem cells into organoid: These assays involve the marking and tracking of infiltrating cells in a way that will distinguish them from the native tissue they are infiltrating. Any dye or label (for example Qtracker (https://www.thermofisher.com/order/catalog/product/Q25021MP?de&en#/Q25021MP?de&en) that penetrates the cells and stays stable and detectable in them for the length of the culturing and assays can be used.

Inflammation (molecular profiling) involves the use of real-time qPCR to detect expression levels of target genetic markers in the cells, which can inform of increased or decreased production of various molecules. Monitoring the transcripts of inflammatory markers can therefore show the molecular inflammatory profile and indicate changes in production of a variety of inflammatory factors.

Reactive oxygen species (ROS): Assays to determine oxidative stress may measure levels of toxic reactive oxygen species or levels of cellular antioxidants. Cells generate ROS as a result of increased metabolism, a stress response or various pathologic states. A variety of assays measuring intracellular or extracellular ROS are available (https://www.thermofisher.com/de/de/home/life-science/ceIl-analysis/cell-viability-and-regulation/oxidative-stress.html).

Reactive nitrogen species quantification: same as for ROS.

Fibrosis assays monitor the excessive deposition of extracellular matrix due to exaggerated repair in response to damage. These can include immunostaining staining for fibrotic markers, real time qPCR (as above) of fibrotic marker transcripts, or others.

Omics technologies are primarily aimed at the universal detection of genes (genomics), mRNA (transcriptomics), proteins (proteomics) and metabolites (metabolomics) in a specific biological sample. (https://www.isaaa.org/resources/publications/pocketk/15/default.asp#:˜:text=Genomics%20provides%20an%20overview%20of,in%20understanding%20organism%27s%20entire%20metabolism; https://obgyn.onlinelibrary.wiley.com/doi/pdf/10.1576/toag.13.3.189.27672).

In certain embodiments, the myeloid cells or lymphoid cells or hematopoietic stem cells comprise a CHIP mutation, wherein the CHIP mutation is a mutation recognized as leading to a clonal hematopoiesis phenotype of the myeloid cells or lymphoid cells or hematopoietic stem cells. The CHIP mutation leads to a clonal hematopoiesis phenotype of the myeloid cells or lymphoid cells or hematopoietic stem cells, meaning that a substantial proportion of mature blood cells is derived from a single dominant hematopoietic stem cell lineage.

In certain embodiments, the self-organized cardiac organoid does not comprise the CHIP mutation.

In certain embodiments, the CHIP mutation is a mutation in a single gene or a combination of genes selected from TET2, DNMT3A, ASXL1, JAK2, SF3B1, SRSF2, TP53, PPM1D, RUNX1, DDX41, U2AF1, IDH1, IDH2, CBL, KRAS, SMC1A, TERT, SH2B3, CHEK2, ATM/PDGFD, PINT, GFI1B, ABCG1, ABCA1, ABCG4, ABCB6, BCOR, BCORL1, GNB1, and GNAS.

In certain embodiments, the CHIP mutation is a mutation in a single gene or a combination of genes selected from TET2, DNMT3A, ASXL1, JAK2, SF3B1, SRSF2, TP53, PPM1D, and RUNX1.

In certain embodiments, the CHIP mutation is a loss-of-function mutation in TET2. In certain embodiments, the CHIP mutation is a loss-of-function mutation or a dominant negative mutation in DNMT3A. In certain embodiments, the CHIP mutation is a gain-of-function mutation or a dominant negative mutation in ASXL1. In certain embodiments, the CHIP mutation is a truncating mutation in ASXL1. In certain embodiments, the CHIP mutation is a gain-of-function mutation in JAK2. In certain embodiments, the CHIP mutation is a gain-of-function mutation in SRSF2. In certain embodiments, the CHIP mutation is a loss-of-function mutation in TP53. In certain embodiments, the CHIP mutation is a gain-of-function mutation in PPM D. In certain embodiments, the CHIP mutation is a loss-of-function mutation or a dominant negative mutation in RUNX1.

### CHIP Mutations

TABLE 1
Gene targets most commonly mutated in CHIP

| Target | Function | Mutation type(s) |
|---|---|---|
| TET2 | Methylcytosine dioxygenase catalyzing | Loss of function |
| | conversion of 5mC to 5hmC | |
| DNMT3A | DNA methyltransferase predominantly | Loss of function |
| | performing de novo genome-wide | Dominant negative |
| | methylation. | Undetermined |
| ASXL1 | Chromatin-binding protein regulating | Gain of function (via |
| | transcription through nuclear hormone | truncated protein) |
| | receptors, such as retinoic acid receptors | Dominant negative |
| | and peroxisome proliferator-activated | Undetermined (multiple |
| | receptor γ. Regulates PR-DUB which | nonsense mutations and |
| | mediates post-translational histone | frameshifts) |
| | modifications | |
| JAK2 | Signaling kinase interacting with a variety of | Gain of function |
| | receptors - prolactin receptor, | (constitutive engagement via |
| | thrombopoietic receptor, and interferon-γ | V617F) |
| | involved in cell growth, development, | |
| | differentiation, and histone modification. | |
| SF3B1 | Component of U2 snRNP which binds to | Undetermined (multiple |
| | pre-mRNA and is involved in RNA splicing | mutation hotspots) |
| | regulation. | |
| SRSF2 | Interacts with pre-mRNA and spliceosomal | Gain of function |
| | components to promote RNA splicing. | |
| TP53 | Tumor suppressor - Transcription factor | Loss of function |
| | regulating cell cycle arrest, apoptosis, | Undetermined |
| | senescence, DNA repair, and metabolism | |
| | changes in response to diverse cellular | |
| | stresses. | |
| PPM1D | A phosphatase, PP2C family member | Gain of function (via |
| | induced by p53, functions to deactivate and | truncated protein) |
| | degrade p53, thus forming a negative | |
| | feedback loop attenuating p53 activation. | |
| RUNX1 | A transcription factor regulating the | Loss of function |
| | differentiation of hematopoietic stem cells | Dominant negative (via |
| | | R174Q) |
| | | Undetermined (via |
| | | translocation and chimeric |
| | | transcripts) |

The listed genetic targets have been associated with CHIP. Due to the field's novelty, there is limited functional data directly linking specific genetic mutations with CHIP.

TABLE 2
A non-exhaustive list of further genes associated with CHIP (with lesser prevalence)

| Target | Extra Information |
|---|---|
| DDX41 | ATP-dependent RNA helicase, required during post-transcriptional gene |
| | expression, involved in pre-mRNA splicing. |
| U2AF1 | Mediates protein-protein and protein-RNA interactions in constitutive and |
| | enhancer-dependent splicing. |
| IDH1 | Isocitrate dehydrogenase, catalyzes oxidative decarboxylation and indirectly |
| | participates in mitigating oxidative damage |
| IDH2 | Paralog of IHD1, catalyzes oxidative decarboxylation |
| CBL | Proto-oncogene, E3 ubiquitin-protein ligase promoting proteasomal degradation, |
| | negative regulator of many signal transduction pathways |
| KRAS | Proto-oncogene with GTPase activity, involved in regulation of cell-proliferation, |
| | involved in oncogenic events by silencing tumor suppressor genes |
| SMC1A | Involved in chromosome cohesion during cell cycle and in DNA repair, central |
| | component of cohesion complex |
| TERT | Telomerase reverse transcriptase, essential for replication of chromosome termini, |
| | has low activity in somatic cells |
| SH2B3 | Tumor suppressor, involved in a range of signaling activities by growth factor and |
| | cytokine receptors, critical in hematopoiesis |
| CHEK2 | Required for checkpoint-mediated cell cycle arrest, activation of DNA repair and |
| | apoptosis in response to DNA double-strand breaks |
| ATM/PDGFD | N/A (Ataxia telangiectasia mutated platelet derived growth factor) |
| PINT | N/A |
| GFI1B | Transcriptional repressor, primarily expressed in hematopoietic cells, controls |
| | genes involved in erythrocyte and megakaryocyte development and maturation |
| ABCG1 | Catalyzes efflux of phospholipids, potential oncogene |
| ABCA1 | Catalyzes translocation of phospholipids, potential oncogene |
| ABCG4 | Involved in cellular cholesterol homeostasis, potential oncogene |
| ABCB6 | Plays a crucial role in heme synthesis, binds heme and porphyrins and functions in |
| | their ATP-dependent update into the mitochondria, potential tumor suppressor |
| BCOR | Transcription corepressor required for germinal center formation, interacts with |
| | BCL6 and some histone deacetylases, may influence apoptosis |
| BCORL1 | Transcriptional corepressor, interacting with histone deacetylases to repress |
| | transcription |
| GNB1 | G protein β subunit, regulator of G protein alpha subunits and other signal |
| | transduction receptors and effectors |
| GNAS | Stimulatory G-protein α subunit involved in transmembrane signal transduction and |
| | modulation. |

The listed genetic targets have been associated with CHIP. Due to the field's novelty, there is limited functional data directly linking specific genetic mutations with CHIP.

In certain embodiments, the myeloid cells are selected from common myeloid progenitor cells, myeloblasts, megakaryocytes, thrombocytes erythrocytes, mast cells, myeloblasts, basophils, neutrophils, eosinophils, monocytes; and macrophages.

In certain embodiments, the lymphoid cells are selected from common lymphoid progenitor cells, small lymphocytes, natural killer cells, small lymphocytes, T-lymphocytes, B-lymphocytes and plasma cells.

In certain embodiments, the myeloid cells or lymphoid cells or hematopoietic stem cells are of different genetic origin than the self-organized cardiac organoid.

### Cell Sources for Cardiac Organoid

patient-derived
induced pluripotent stem cell (iPSCs)—derived
embryonic stem cell (ESCs)—derived
Adult stem and pluripotent cells
   Hematopoietic stem cells
   Hematopoietic pluripotent and progenitor cells
   Mesenchymal stem cells
   Others
established immortalized cell lines
Somatic cell nuclear transfer (SCNT)—derived cells

### Cell Sources for Myeloid/Lymphoid/Hematopoietic Stem Cells

patient-derived
induced pluripotent stem cell (iPSCs)—derived
embryonic stem cell (ESCs)—derived
Adult stem and pluripotent cells
   Hematopoietic stem cells
   Hematopoietic pluripotent and progenitor cells
   Mesenchymal stem cells
   Others
established immortalized cell lines
Somatic cell nuclear transfer (SCNT)—derived cells

In certain embodiments, the pharmaceutical compound is applied to the myeloid cells or the lymphoid cells or hematopoietic stem cells, before they are contacted with the cardiac organoid.

In certain embodiments, the pharmaceutical compound is applied to the cardiac cell culture model system after step ii.

In certain embodiments, the myeloid cells or the lymphoid cells or hematopoietic stem cells are labelled with a dye before step ii. In certain embodiments, the myeloid cells or the lymphoid cells or hematopoietic stem cells are labelled with a fluorescent dye before step ii. In certain embodiments, the myeloid cells or the lymphoid cells or hematopoietic stem cells are labelled with a Qtracker cell dye (https://www.fishersci.de/shop/products/molecular-probes-qtracker-qdot-525-cell-labelinq-kit/10232933). General infos on cell dyes can be found under https://www.thermofisher.com/us/en/home/life-science/cell-analysis/cell-tracing-tracking-and-morphology/cell-tracking.html.

In certain embodiments, the cardiac cell culture model system is a model for a disease selected from:
clonal hematopoiesis of indeterminate potential (CHIP);
myocarditis;
myocardial inflammation;
viral or bacterial or parasitic infectious disease;
sepsis; and
transplant tissue rejection.

Myocarditis can result from infectious and noninfectious cases:

Infectious: can be caused by viral (adenoviruses, enteroviruses, parvovirus B19, Herpesviridae, HIV, HepC and Influenza A and B, Coronaviridae etc), bacteria (such as Borrelia spp.), protozoa (such as Trypanosoma cruzi) and fungi.

For these, the system plus the infectious agents are used to model the infectious disease, to assess the effects of the disease on various cardiac genetic backgrounds; to assess the effects of the infectious disease on various lymphoid/myeloid/hematopoietic stem cell genetic backgrounds, to assess the effects of drugs in mediating these pathologies for drug discovery.

Noninfectious: can be induced by a wide variety of toxic substances and drugs (such as immune checkpoint inhibitors) and systemic immune-mediated diseases, autoimmune diseases, congenital/genetic causes etc.

For these, the system is used by genetically modifying the invading cells to create a model of the specific systemic immune, autoimmune, or congenital disease, with the specific genes and alterations that pertain to that disease state, and then assess as described before.

For toxicity and drug-induced myocarditis, the system is used with the toxic substances to induce these disease states to model the disease, to assess the effects of the disease state in various cardiac genetic backgrounds; to assess the effects of the disease state in various lymphoid/myeloid/hematopoietic stem cell genetic backgrounds, to assess the effects of drugs in mediating these pathologies for drug discovery with various cardiac and/or lymphoid/myeloid/hematopoietic stem cell genetic backgrounds.

In certain embodiments, step iii of maintaining said cardiac cell culture model system under conditions of cell culture is performed under ˜1% O₂.

In certain embodiments, 3000-8000 cells selected from myeloid cells, lymphoid cells, or hematopoietic stem cells, are added per cardiac organoid. In certain embodiments, ˜5000 cells selected from myeloid cells, lymphoid cells, or hematopoietic stem cells, are added per cardiac organoid.

In certain embodiments, the cardiac organoid is cultured for ˜35 days before step ii.

In certain embodiments, the cells selected from myeloid cells, lymphoid cells, or hematopoietic stem cells, are passaged for 4 to 5 times before step ii.

In certain embodiments, step iii of maintaining said cardiac cell culture model system under conditions of cell culture lasts 36 to 72 hours before the read-out is detected in step iv. In certain embodiments, step iii of maintaining said cardiac cell culture model system under conditions of cell culture lasts ˜48 hours before the read-out is detected in step iv.

A second aspect of the invention relates to a cardiac cell culture model system as described in the first aspect and its embodiments.

In certain embodiments of the second aspect, the myeloid cells or lymphoid cells or hematopoietic stem cells comprise a CHIP mutation.

### Features of the Cardiac Cell Culture Model System

In certain embodiments, key system features are that the system includes: (1) having the aforementioned CHIP genes knocked out, knocked down, or otherwise edited in the blood cells, (2) support organoid culturing (such as u-bottom, low attachment multiwell plates) by featuring both entities (blood cells and organoids) in the same culture/medium compatible with maintaining them both, and enabling selective tracking of the infiltrating cells to be able to distinguish them from native cardiac organoid ones.

Several discovery and optimization steps were beneficial for the system to function:
1) A precisely targeted number of lymphoid/myeloid/hematopoietic stem cell cells.
2) A precisely timed monitoring and collection of data from the system.
3) The environmental conditions for culturing of this system had to be identified; The inventors have reached the conclusion that 1% O₂ is necessary for the system to work properly.
4) Imaging data collection was improved from conventional approaches; Rather than sampling a few locations of the tissue, the inventors cut and image through every section of the organoid, creating a 3D reconstruction of the organoid and the infiltrating cells; The sum of these is necessary for a complete view of the status of the system and the degree of pathology and is not achievable with conventional representative image slices.

Wherever alternatives for single separable features such as, for example, a cell type or a mutation are laid out herein as “embodiments”, it is to be understood that such alternatives may be combined freely to form discrete embodiments of the invention disclosed herein. Thus, any of the alternative embodiments for a cell type may be combined with any of the alternative embodiments of a mutation and these combinations may be combined with any read-out method mentioned herein.

### Items

1. A method for pharmaceutical compound screening comprising the steps
   i. providing
      a. a cardiac organoid comprising
         cardiomyocytes
   ii. contacting said cardiac organoid with
      b. myeloid cells,
      lymphoid cells, or
      hematopoietic stem cells;
   thereby yielding a cardiac cell culture model system,
   iii. maintaining said cardiac cell culture model system under conditions of cell culture;
   wherein a pharmaceutical compound of interest is contacted with
      the myeloid cells or the lymphoid cells or the hematopoietic stem cells, before they are contacted with the cardiac organoid; or
      the cardiac cell culture model system after step ii,
   iv. detecting a read-out of the effect of said pharmaceutical compound on the cardiac cell culture model system,
   v. optionally, repeating steps i-iv with a different pharmaceutical compound.
2. The method according to item 1, wherein the cardiac organoid is a cardiomyocyte monoculture comprising:
   cardiomyocytes.
   3. The method according to item 1, wherein the cardiac organoid is a cardiomyocyte biculture comprising:
   cardiomyocytes; and
   fibroblasts.
   4. The method according to item 1, wherein the cardiac organoid is a triculture comprising:
      cardiomyocytes;
      endothelial cells; and
      fibroblasts.
   5. The method according to item 1, wherein the cardiac organoid is a self-organized cardiac organoid comprising
      epicardium;
      myocardium;
      endocardium; and
      cardiac lumen,
   wherein said self-organized cardiac organoid comprises:
      cardiomyocytes;
      endothelial cells;
      fibroblasts;
      smooth muscle cells;
      pericytes;
      neurons/sino-atrial node cells.
   6. The method according to any one of the preceding items 1 to 5, wherein the read-out is detected as cell viability of the cardiac organoid.
   7. The method according to any one of the preceding items 1 to 5, wherein the read-out is detected as cell viability of myeloid cells or lymphoid cells or hematopoietic stem cells.
   8. The method according to any one of the preceding items 1 to 5, wherein the read-out is detected as cytotoxicity of the lymphoid cells onto the cardiac organoid.
   9. The method according to any one of the preceding items 1 to 5, wherein the read-out is detected as cardiomyocyte cell death.
   10. The method according to any one of the preceding items 1 to 5, wherein the read-out is detected as proliferation of the cells of the cardiac organoid.
   11. The method according to any one of the preceding items 1 to 5, wherein the read-out is detected as proliferation of the myeloid cells or lymphoid cells or hematopoietic stem cells.
   12. The method according to any one of the preceding items 1 to 5, wherein the read-out is detected as proliferation of macrophages.
   13. The method according to any one of the preceding items 1 to 5, wherein the read-out is detected as contractility.
   14. The method according to any one of the preceding items 1 to 5, wherein the read-out is detected as mitochondrial activity.
   15. The method according to any one of the preceding items 1 to 5, wherein the read-out is detected as metabolism.
   16. The method according to any one of the preceding items 1 to 5, wherein the read-out is detected as cell infiltration of the myeloid or lymphoid or hematopoietic stem cells into organoid.
   17. The method according to any one of the preceding items 1 to 5, wherein the read-out is detected as inflammation.
   18. The method according to any one of the preceding items 1 to 5, wherein the read-out is detected as reactive oxygen species quantification.
   19. The method according to any one of the preceding items 1 to 5, wherein the read-out is detected as reactive nitrogen species quantification.
   20. The method according to any one of the preceding items 1 to 5, wherein the read-out is detected as fibrosis of the cardiac organoid.
   21. The method according to any one of the preceding items 1 to 5, wherein the read-out is detected as genomics.
   22. The method according to any one of the preceding items 1 to 5, wherein the read-out is detected as transcriptomics.
   23. The method according to any one of the preceding items 1 to 5, wherein the read-out is detected as proteomics.
   24. The method according to any one of the preceding items 1 to 5, wherein the read-out is detected as metabolomics.
   25. The method according to any one of the preceding items, wherein the myeloid cells or lymphoid cells or hematopoietic stem cells comprise a CHIP mutation, which leads to a clonal hematopoiesis phenotype of the myeloid cells or lymphoid cells or hematopoietic stem cells, meaning that a substantial proportion of mature blood cells is derived from a single dominant hematopoietic stem cell lineage.
   26. The method according to item 25, wherein the self-organized cardiac organoid does not comprise said CHIP mutation.
   27. The method according to any one of the preceding items 25 to 26, wherein said CHIP mutation is a mutation in a single gene or a combination of genes selected from TET2, DNMT3A, ASXL1, JAK2, SF3B1, SRSF2, TPS3, PPM1D, RUNX1, DDX41, U2AF1, IDH1, IDH2, CBL, KRAS, SMC1A, TERT, SH2B3, CHEK2, ATM/PDGFD, PINT, GFI11B, ABCG1, ABCA1, ABCG4, ABCB6, BCOR, BCORL1, GNB1, and GNAS.
   28. The method according to any one of the preceding items, wherein the myeloid cells are selected from common myeloid progenitor cells, myeloblasts, megakaryocytes, thrombocytes erythrocytes, mast cells, myeloblasts, basophils, neutrophils, eosinophils, monocytes and macrophages.
   29. The method according to any one of the preceding items 1 to 27, wherein the lymphoid cells are selected from common lymphoid progenitor cells, small lymphocytes, natural killer cells, small lymphocytes, T-lymphocytes, B-lymphocytes and plasma cells.
   30. The method according to any one of the preceding items, wherein the myeloid cells or lymphoid cells or hematopoietic stem cells are of different genetic origin than the self-organized cardiac organoid.
   31. The method according to any one of the preceding items, wherein the pharmaceutical compound is applied to the myeloid cells or the lymphoid cells or hematopoietic stem cells, before they are contacted with the cardiac organoid.
   32. The method according to any one of the preceding items, wherein the pharmaceutical compound is applied to the cardiac cell culture model system after step ii.
   33. The method according to any one of the preceding items, wherein the cardiac cell culture model system is a model for a disease selected from:
      clonal hematopoiesis of indeterminate potential (CHIP);
      myocarditis;
      myocardial inflammation;
      viral or bacterial or parasitic infectious disease;
      sepsis; and
      transplant tissue rejection.
   34. A cardiac cell culture model system as described in any one of the preceding items.

The invention is further illustrated by the following examples and figures, from which further embodiments and advantages can be drawn. These examples are meant to illustrate the invention but not to limit its scope.

### DESCRIPTION OF THE FIGURES

FIG. 1 shows the scheme of the invention.

FIG. 2 shows efficient knockdown. Relative levels of TET2 mRNA decreased post-transfection, indicating successful TET2 knockdown. qPCR measurements were performed on Control and TET2 KD macrophages. Relative levels of TET2 mRNA are decreased post-transfection in the TET2 KD group, indicating a successful TET2 knockdown and model establishment.

FIG. 3 shows effective cell labelling and macrophage infiltration. Tracked/Stained Macrophages infiltrate cardiac organoid. The staining and subsequent tracking of Macrophages enables monitoring and analysis of their infiltration into cardiac organoids.

FIG. 4 shows higher infiltration of TET2-deficient macrophages into cardiac tissue under cardiopathology conditions (TET2 KD). Monitoring of macrophage infiltration into organoids shows increased infiltration of TET2 KD macrophages as compared to Control, replicating the increased macrophage infiltration observed in CHIP patients.

FIG. 5 shows evident macrophage infiltration in organoids. Infiltration areas are devoid of cardiomyocytes. Notable macrophage clustering is visible. 3D rendering of organoids with labeled infiltrating macrophages. The absence of cardiomyocytes from infiltration areas further confirms effective macrophage staining and tracking. Additionally, macrophage clustering is demonstrated.

FIG. 6 shows quantification of macrophage infiltration in organoids. Increased area covered with macrophages in TET2 KD organoids (left). Increased macrophage markers present in organoids (right). Quantification of macrophage infiltration in organoids shows increased organoid area covered with macrophages in organoids cocultured with TET2 KD macrophages compared to Control (left). qPCR measurements show increased macrophage markers present in organoids co-cultured with TET2 KD macrophages compared to Control (right).

FIG. 7 shows increased inflammatory and fibrotic marker levels in TET2 KD in macrophages. qPCR measurements show increased inflammatory marker levels (IL-1B, IL6, CCL-5, IL18) in TET2 KD macrophages compared to Control, demonstrating increased inflammation in TET2 KD model, matching the inflammation phenotype in CHIP patients. In addition, they show increased fibrotic marker levels (NPPB, Col1a1, Col2a1, Col3a1, Col4a1, CTGF), demonstrating increased cardiac damage and initiation of fibrotic processes.

FIG. 8 shows cardiomyocyte apoptosis. Colocalization of cardiomyocytes (CMs) and cleaved caspase staining demonstrates it is the CMs that undergo apoptosis. Evident colocalization of cardiomyocytes (CMs) and Cleaved-Caspase 3 apoptosis staining demonstrates that it is the CMs that undergo apoptosis.

FIG. 9 shows TET2-deficient macrophages induce higher levels of cardiomyocyte apoptosis under cardiopathology conditions.

FIG. 10 shows that cardiomyocyte apoptosis quantification shows increase under TET2 KD conditions, as measured by cleaved caspase and cTNT assays. Quantification of cleaved caspase 3 staining shows increased apoptosis in the TET2 KD group as compared to control (left). The increased cardiomyocyte death is further confirmed by an increase in the prognostic cardiac biomarker cTNT in the TET2 KD group.

FIG. 11 shows synthetic lethality screen of a drug library for selective elimination of TET2-monocytes led to a selection of 9 hits that selectively inhibit TET2-deficient cells. Synthetic lethality screen of a drug library highlighted 9 hits that selectively inhibit TET2 KD cells. This demonstrates the use of this platform for drug screening and development for CHIP and related indications.

FIG. 12 shows synthetic lethality screen of a drug library for selective elimination of TET2-monocytes led to the elucidation of multiple drugs that favor the proliferation of TET2-monocytes and exacerbate the effects of CHIP. Synthetic lethality screen of a drug library highlighted inverse hits, i.e. drugs that favor the proliferation of TET2 KD monocytes and exacerbate the effects of CHIP. This demonstrates the use of this platform for drug screening for contraindicated drugs for patients with CHIP and related indications.

FIG. 13 shows immunostaining of organoids (DAPI, α-Actinin) and macrophages (Qtracker) showing limited infiltration and interaction of macrophages in organoids at 20% O₂. In contrast, culturing at 1% O₂ shows further increases of infiltration, thus perfectly recapitulating the expected phenotype as found in human patients.

### EXAMPLES

### Results

### Example 1

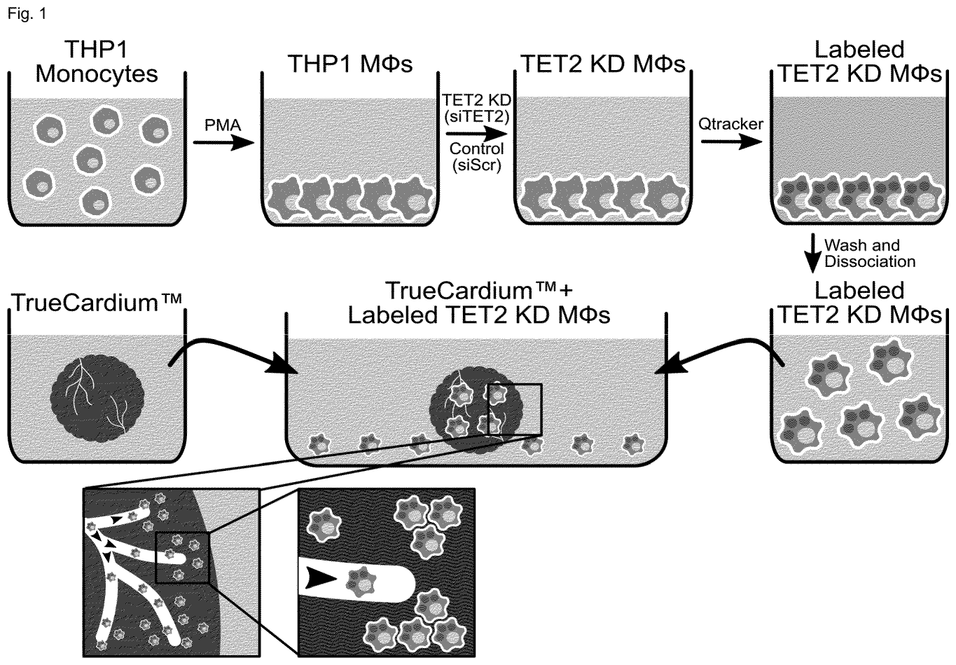

The invention involves the coculturing of lymphoid/myeloid/hematopoietic stem cells with cardiac organoids (FIG. 1).

The description below refers to the THP1-derived macrophages used in the TET2 KD example model.

The cells are cultured, and if necessary, differentiated to the respective target lineage. In this case, THP1 monocytes are differentiated to Macrophages using phorbol 12-myristate 13-acetate (PMA). They are then genetically modified to display the desired CHIP phenotype (in this case with siRNA against TET2 which results in a TET2 KD). The cells are then labeled by incubation with the tracker, after which they are washed and dissociated.

The organoid and myeloid/lymphoid/hematopoietic stem cells are added together in a culturing low-attachment U-bottom multiwell cell culture plate and cultured in 1:1 RPMI/Maintenance media Culturing conditions: 1% O₂, 5% CO₂, 37° C.; They are cultured for 66 hrs before fixing/imaging;

### Caveats/Multi-Options:

1: The myeloid/lymphoid/hematopoietic stem cells are either cultured with appropriate methods to reach the necessary stage before addition to the system, OR added to the system directly
2: Drugs for Screening can be added to the coculturing OR to just the organoid or just the I/m/hsc cells before addition to the system
3: the genetic modification can be via siRNA, via CRISPR, or via other methods.
4: The cells can be engineered before or after differentiation, or at any point in the differentiation/development.

### Example 2

Clonal Hematopoiesis of Indeterminate Potential (CHIP) arises as a result of acquired mutations in a genetic allele in somatic cells, particularly hematopoietic stem cells and all their progeny cell types. It occurs when the acquired mutations lead to a proliferative or survival advantage of the cells carrying it, as well as their progeny. This causes a clonal expansion of the mutation-carrying cells, leading to them taking up a disproportionately large proportion of the cells in the blood.

CHIP has been associated with increased risk and development of cardiovascular disease (such as coronary heart disease, early onset myocardial infarction, etc.), and it is a predictor of mortality, independent of other risk factors. Given that the cause of the issue lies in cells from the blood and the affected tissue is cardiac, it follows that the phenotype arises from the interaction of the two. In particular, the infiltration of myeloid and lymphoid cells into the cardiac tissue and their interactions with it thereafter are the principal causes of the cardiac pathology. The interactions include increased cardiomyocyte (CM) death, inflammation and cardiac tissue fibrosis and others. This type of interaction is crucial for the development not only of CHIP-based cardiopathology, but also of various types of myocardial inflammation and myocarditis (infectious, congenital, immune-mediated, autoimmune and other types), sepsis, transplant tissue rejection and others.

The inventors aimed to develop a system for disease modeling and subsequent drug screening, validation, and development. To create such disease models, several essential elements are needed. Firstly, given that the pathology arises in the adult organism, adult matured cardiac tissue must be used, so that it recapitulated the structural and functional properties of the adult organism, and so that it replicates the physical, biochemical, molecular and physiologic responses of the native human heart. Secondly, both entities, i.e. the HSC/myeloid/lymphoid cells as well as the cardiac tissue must not only coexist but also interact to replicate the pathology in vitro. Thirdly, the system must allow for the selective tracking and evaluating of the two entities and their interactions in order to assess the effects of genetic background, environmental cues, drug treatment and other stimuli on both the development and on the amelioration of the pathology. Fourthly, CHIP-associated genetic modifications of the infiltrating cells need to be done so that the system fully recapitulates the CHIP pathology.

No system exists to date that encapsulates the necessary points.

Therefore, the inventors have created a system that satisfies these crucial points and successfully functions as a model for CHIP and related indications.

### Example 3

Firstly, the inventors are using a state-of-the-art 3D cardiac organoid technology which displays properties of mature adult cardiac tissue. It also responds to cues in a physical, biochemical, molecular and physiologic manner equivalent to the mature adult cardiac tissue. Secondly, the inventors have optimized a coculturing system that enables the infiltration of the myeloid/lymphoid/hematopoietic stem cells into the cardiac organoids and the subsequent meaningful interaction of the two entities. Thirdly, the inventors have established methods for selective tracking of the infiltrating cells, and a portfolio of assays evaluating the interactions between the two entities, and consequent drawing of meaningful and relevant conclusions regarding the effects of genetic background, environmental cues, drug treatment and other stimuli on the development and amelioration of the pathology. The system up to this point reflects the physiological state of an adult organism. Upon genetic modification of the myeloid/lymphoid/hematopoietic stem cells with CHIP-associated mutations, the system reflects the physiological state of an organism with CHIP.

### Example 4

To demonstrate the recapitulation of CHIP with this system, the inventors have chosen to implement a TET2-based CHIP model. TET2 is one of the most frequently mutated genes in CHIP patients. The mutations occur mostly on a single genetic allele, and despite the variety in mutation types detected in CHIP patients, they all lead to a loss of function of the gene product.

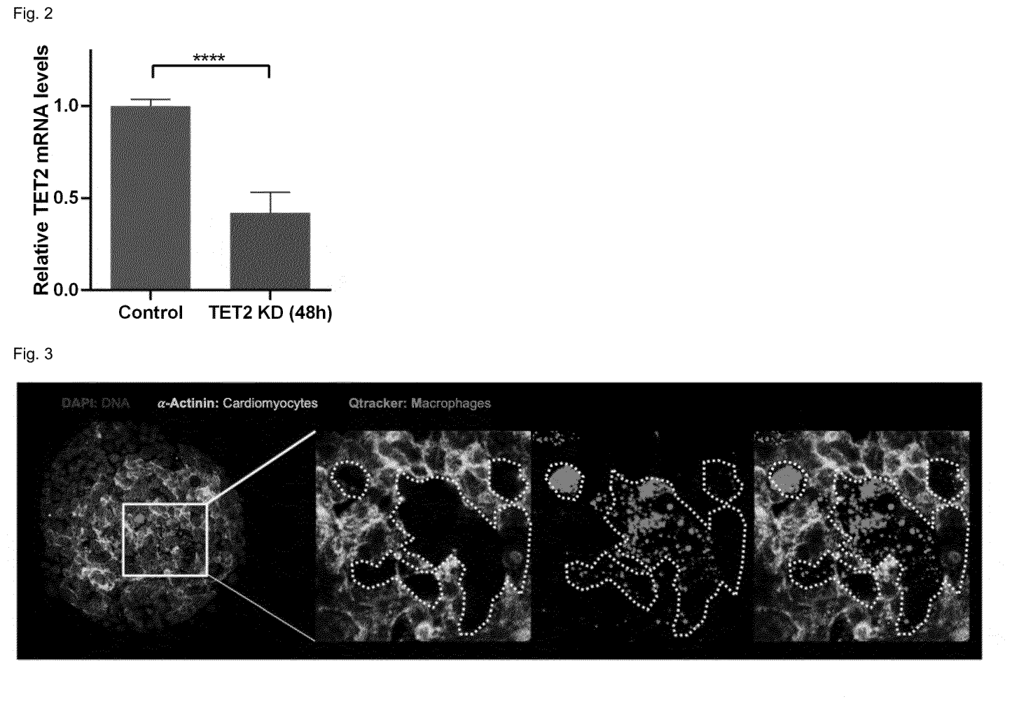

The inventors have used siRNA to knock down TET2 (TET2 KD) in THP macrophages (MΦs). FIG. 2 shows qPCR measurements of relative TET2 mRNA levels in control and TET2 KD MΦs. The relative levels of TET2 mRNA are significantly decreased post-transfection in the TET2KD group (** p<0.0001), indicating a successful TET2 knockdown, replicating the aforementioned loss of function.

These MΦs were utilized in the coculturing system with self-organized human cardiac organoids, where they were tracked using a Qtracker stain. FIG. 3 shows images of MΦs in the cardiac organoid, demonstrating successful selective tracking of the infiltrating cells, enabling monitoring and analysis of the degree of infiltration, their location in the organoid and other parameters.

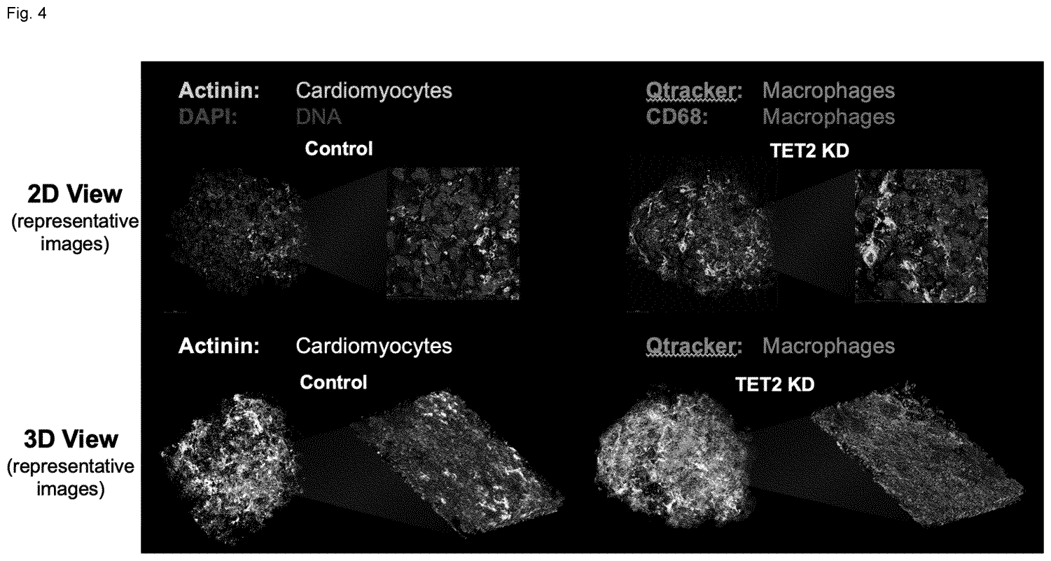

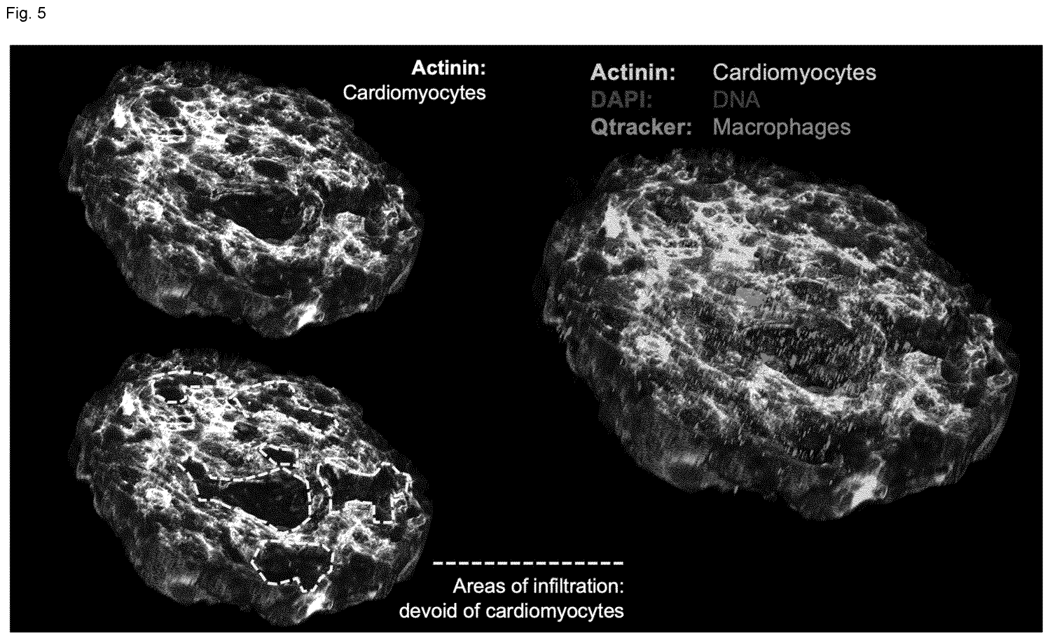

When control and TET2 KD MΦs were allowed to infiltrate cardiac organoids in cardiopathologic conditions, a higher infiltration of TET2 KD MΦs was observed than of control MΦs, as the representative images in FIG. 4 show. This replicates the increased MΦs infiltration observed in CHIP patients. FIG. 5 shows a 3D rendering of organoids with labeled infiltrating MΦs. The absence of CMs from infiltration areas further confirms effective MΦs straining and tracking. Additionally, notable MΦs clustering is visible.

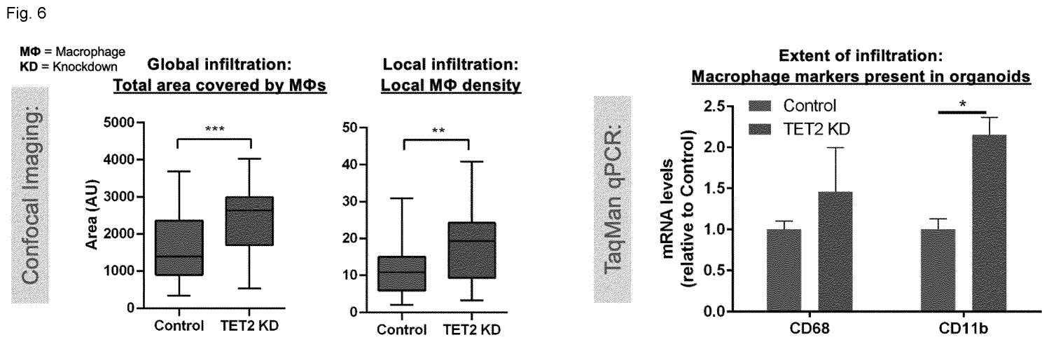

The inventors quantified the levels of MΦs infiltration in organoids. As shown in FIG. 6, the area covered by MΦs is increased when TET2 KD MΦs infiltrate an organoid compared to control cells (30+ regions of interest from multiple different organoids assessed, ** P<0.001, * p<0.01). qPCR measurements of Mm marker RNA show increased expression of Mm markers CD68 and CD11b in organoids infiltrated by TET2 KD MΦs than control ones, indicating increased infiltration potential of the TET2 KD cells (* p<0.05).

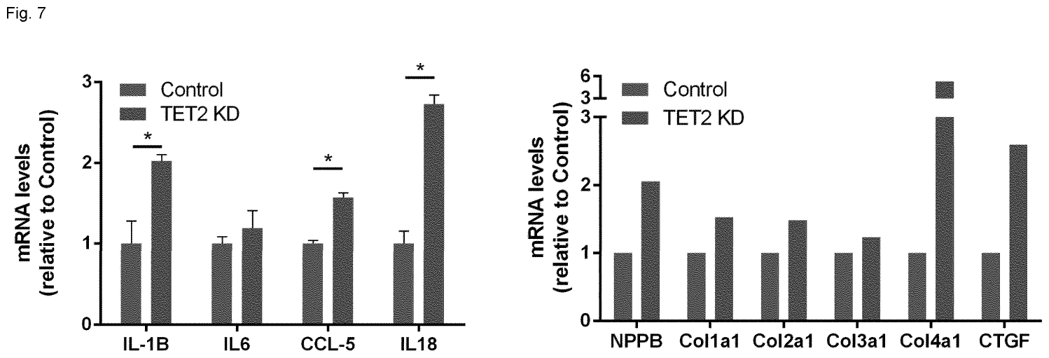

FIG. 7 shows increased relative mRNA levels of inflammatory markers IL-1B, IL6, CCL-5, and IL18 in TET2 KD MΦs compared to control MΦs (n=2, *p<0.05). This demonstrates increased inflammation in the TET2 KD model, matching the inflammation phenotype observed in CHIP patients. In addition, relative mRNA levels of fibrotic marker (NPPB, Col1a1, Col2a1, Col3a1, Col4a1, CTGF) are also increased in TET2 KD compared to control, demonstrating increased cardiac damage and initiation of fibrotic processes. This indicates increased fibrosis, which is an indicator of tissue damage and the body's attempt to make up for the cell loss by fibroblast expansion, which ends up compromising the function of the heart.

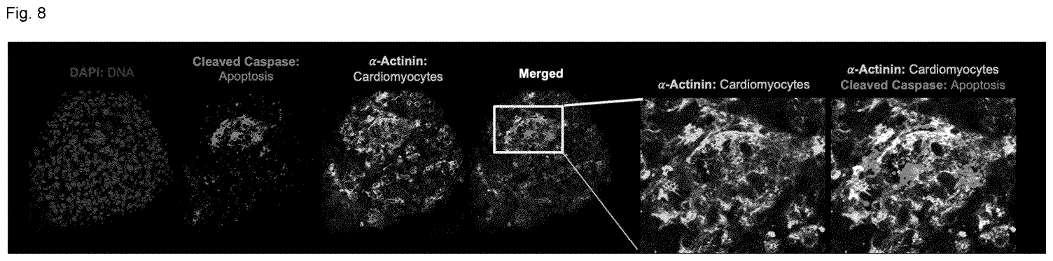

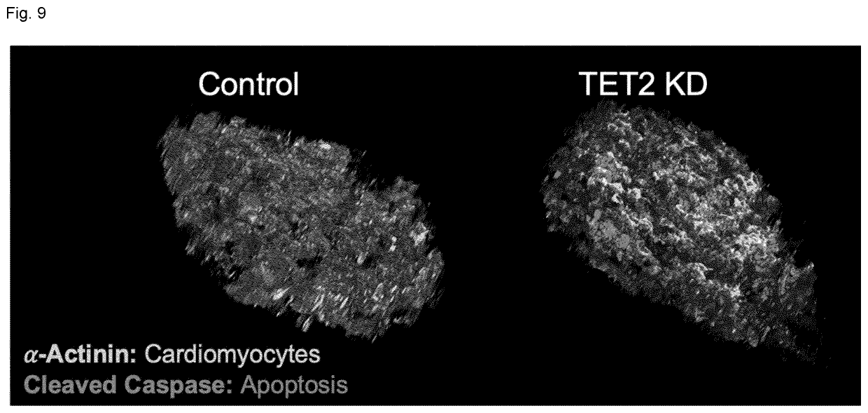

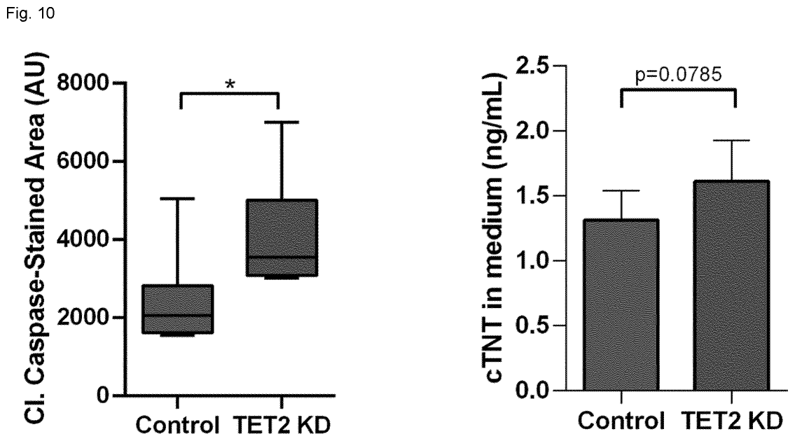

To assess the CM death, the inventors used cleaved caspase staining which labels apoptotic cells. The colocalization of this staining and the α-Actinin staining (for CMs) shown in FIG. 8 indicates that it is indeed CMs that undergo apoptosis. FIG. 9 shows a 3D rendering where it is evident that organoids infiltrated by TET2 KD MΦs experience higher levels of CM apoptosis compared to those infiltrated by control cells. FIG. 10 shows the quantification of area covered by cleaved caspase 3, and an evident increase of CM apoptosis in organoids infiltrated with TET2 KD MDs (* p<0.05). The increase in CM death in the TET2 KD group is further verified by an increase in cardiac troponin T (cTNT), a prognostic cardiac damage clinical biomarker. These indicators together demonstrate that this model recapitulates the cardiomyocyte loss observed in CHIP patients.

In summary, increased infiltration, inflammation and cardiomyocyte death have been accurately recapitulated by this disease model utilizing the invention.

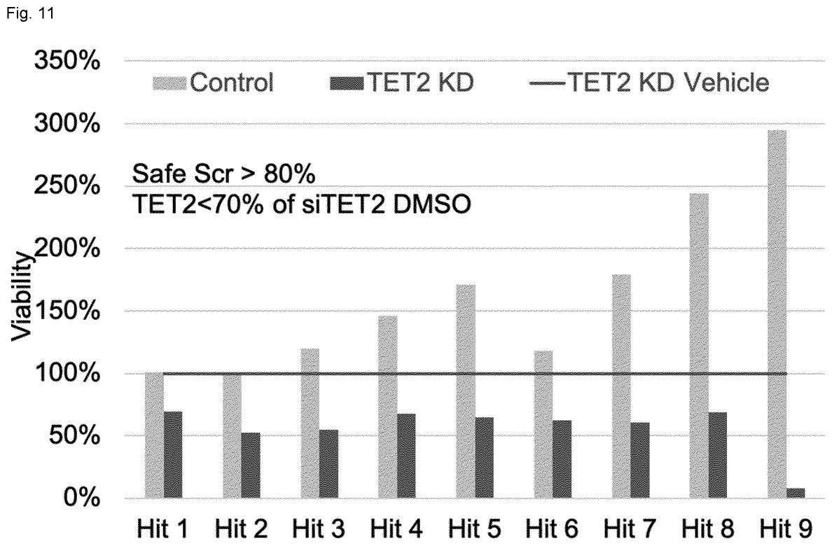

The established disease model can subsequently be used for drug screening. Thus, a synthetic lethality screening of a drug library was performed, treating TET2 KD or control cells. FIG. 11 shows 9 hits highlighted by the drug screen which selectively inhibit TET2 KD cell viability, and can therefore be useful in treating CHIP. This demonstrates the use of this system for drug screening and development for CHIP and related indications.

### Example 5

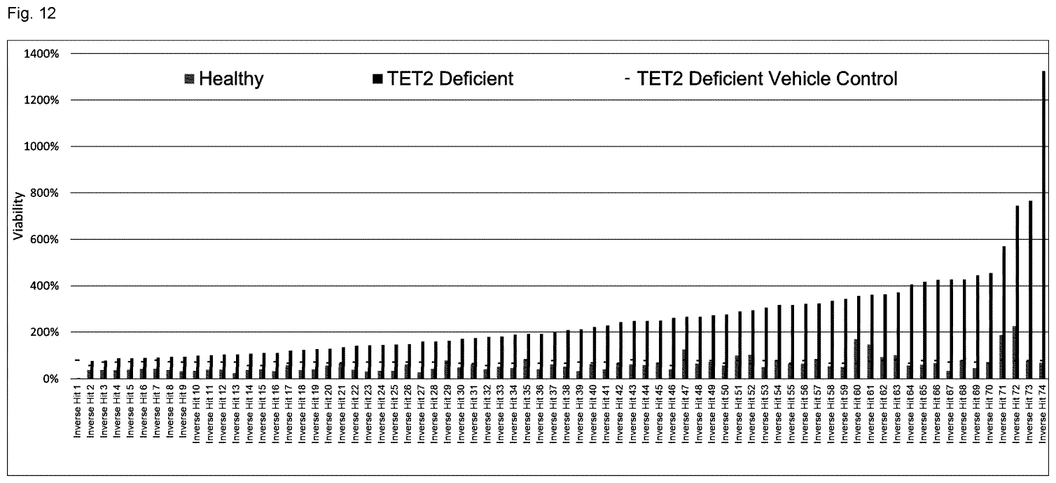

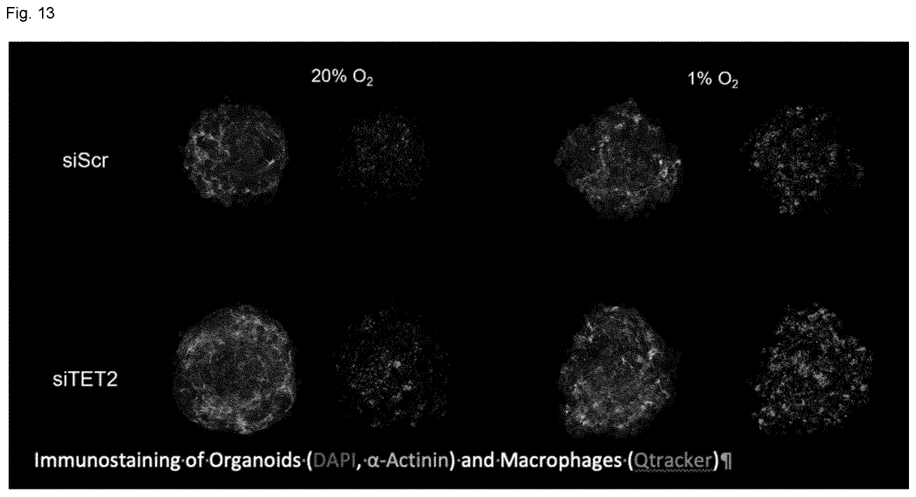

Additionally, the synthetic lethality screen highlighted inverse hits, i.e. drugs that favor the proliferation of TET2 KD cells and exacerbate the severity of CHIP. FIG. 12 shows 74 such drugs. This demonstrates the use of this system for drug screening for contraindicated drugs for patients with CHIP and related indications.

In addition, the information on drugs suppressing or increasing TET2 KD cell viability can be used to further investigate the mechanisms involved in chip development and aid in target discovery and development of targeted therapies.

### Example 6

Culturing in different O₂ concentrations was tested: 20% and 1%. The culturing in 20% gave an insufficient baseline infiltration and coverage of macrophages in the organoid. The interactions between the infiltrating cells and the cardiac organoid are essential for the model, and therefore 1% O₂ was selected as a dependable condition with adequate cell-organoid interactions (FIG. 13).

### Material and Methods

Material

| | | |
|---|---|---|
| TeSR ™-E8 ™ Kit | Stemcell | Cat 05940 |
| | Technologies | |
| * RPMI 1640 with Glutamax | Invitrogen | Cat 61870-010 |
| * Endothelial Cell Growth | Promocell | Cat C-22111 |
| Medium 2 kit | | |
| 100 mM sodium pyruvate | Invitrogen | Cat 11360 |
| 100X penicillin/streptomycin | Invitrogen | Cat 15140 |
| BMP4 | R&D systems | Cat 314-BP |
| Activin A | R&D systems | Cat 338-AC |
| FGF-2 | Miltenyi | Cat 130-093-841 |
| | Biotech | |
| VEGF | R&D systems | Cat 293-VE |
| IWP4 | Stemgent | Cat 04-0036 |
| CHIR99021 | Stemgent | Cat 04-0004 |
| Rock Inhibitor Y27632 | Stemcell | Cat 72302 |
| | Technologies | |
| B27 | Gibco | Cat 17504044 |
| DMSO | Sigma | Cat D2650 |
| L-ascorbic acid 2 phosphate | Sigma | Cat A8960-5G |
| sesquimagnesium salt | | |
| hydrate (ASC), | | |
| Matrigel | Corning | Cat 354277 |

* Endothelial Cell Growth Medium 2 contains (in 500 mL of medium - information available https://www.promocell.com/f/product-information/manual/C-22111.pdf):
Epidermal Growth Factor (recombinant human) 5 ng/ml
Basic Fibroblast Growth Factor (recombinant human) 10 ng/ml
Insulin-like Growth Factor (R3 IGF-1) 20 ng/ml
Vascular Endothelial Growth Factor 165 (recombinant human) 0.5 ng/ml
Ascorbic Acid 1 μg/ml
Heparin 22.5 μg/ml
Hydrocortisone 0.2 μg/m
* RPMI 1640 with Glutamax contains (in 500 mL of medium - information available https://www.thermofisher.com/de/de/home/technical-resources/media-formulation.122.html)

### Organoid Culturing

Human induced pluripotent stem cells (hiPSCs) were used for TrueCardium generation. In brief, 500 hiPSCs were cultured on ultra-low-attachment surface in medium TeSR™-E8™ at 37° C. and 5% CO₂ at humidified atmosphere to form embryoid body (EB). After 2 days, EBs were differentiated to cardiac organoids (COs) by replacing medium every 48 hours with medium 1, medium 2, medium 3 (2 times) and medium 4. COs were maintained in medium 4 for 10 days by refreshing medium every second day. Medium 4 was then replaced by medium 5 for 4 days with refreshing medium every 48 h. After that COs were transferred to rotating incubator with medium 6 for 6 days with refreshing medium every second day. COs were then ready to be harvested.

Media info:
Basal medium:
   RPMI 1640 with Glutamax
   1% of 100× sodium pyruvate
   1% of 100× penicillin/streptomycin
   3% B27 supplement
Medium 1:
   Basal medium
   50 ng/mL of Activin A
   2 ng/mL of BMP-4
   5 ng/mL of hFGF
   1 μM CHIR
   100 μM ACS-2-P
Medium 2:
   Basal medium
   5 ng/mL of Activin A
   10 ng/mL of BMP-4
   5 ng/mL of bFGF
   1 μM CHIR
   100 μM ACS-2-P
Medium 3:
   Basal medium
   5 μM IWP4
   200 μM ACS-2-P
Medium 4:
   Basal medium
   200 μM ACS-2-P

### Cell Density of iPS Cells in Cardiac Organoid:

Optimize from 500 to 3000 iPSCs per EB
Density of 500 iPSCs per EB was chosen as it gave best result in organoid formation.

### Timing for the Cardiac Organoid Generation:

Day −2: EB formation from iPSCs in microwell
Day 0: Add Medium 1 supplemented with Matrigel PSC grade (dilution 1:100)
After exact 48 h (day 2): Add Medium 2
After exact 48 h (day 4): Add Medium 3
After exact 48 h (day 6): Change fresh Medium 3
After exact 48 h (day 8): Add Medium 4
From day 8 to day 18: Change fresh Medium 4 every second day
Day 18: Add Medium 4 supplemented with 50 nM hVEGF and 25 nM hFGF
After exact 48 h (day 20): Change fresh Medium 4 supplemented with 50 nM hVEGF and 25 nM hFGF
After exact 48 h (day 22): transfer Cardiac organoids to rotating incubator and add Mix Medium (Medium 4: EGM2 (without FBS) at ratio 4:1)
From day 22 to day 28: maintain cardiac organoids in rotating incubator. Change mix medium every second day
Day 29: Harvest cardiac organoid

### Macrophage Culturing

THP-1 cells were purchased from DSMZ (ACC 16) and maintained in RPMI-1640 (Gibco)+10% Fetal Calf Serum, 1% Penicillin/Streptomycin, 1% Glutamine, 0.05 mM 2-Mercaptoethanol.

THP-1 cells were differentiated from monocytes to macrophages by supplementing the media 100 ng/mL PMA and culturing for 48 h, and then in PMA-free media for another 24 h.

For routine culturing, cells were maintained at 37° C., 5% CO₂, 20% O₂. In cardiopathologic culturing, both THP1 cells and organoids were maintained hypoxic conditions at 37° C., 5% CO₂, 1% 02.

### TET2 Knockdown

Transfection of THP-1 cells with siRNA purchased as ON-TARGETplus pools from Horizon Discovery (TET2 siRNA pool containing: ACAAGAAAGUAGAGGGUAU (SEQ ID 001), ACACCUAGUUUCAGAGAAU (SEQ ID 002), CCUCAGAAUAAUUGUGUGA (SEQ ID 003), CAGCAAAGGUACUUGAUAC (SEQ ID 004); Control siRNA pool containing: UGGUUUACAUGUUGUGUGA (SEQ ID 005), UGGUUUACAUGUUUUCCUA (SEQ ID 006)).

Working solution of 50 μM siRNA was prepared in siRNA Buffer (ThermoFisher). 2 μL of 50 μM siRNA were mixed with 500 μL OptiMEM and 6.25 μL LTX Reagent PLUS (ThermoFisher) and incubated at room temperature (RT) for 15 min. 6.25 μL Lipofectamine LTX was added, mixed, and incubated for 30 min at RT. 500 μL were then added to the target culture and the culture was incubated at 37° C., 5% CO₂. Passaging and/or media change was performed after 24 h.

### Organoid and Macrophage Coculture

Macrophages were differentiated from THP1 monocytes, then treated with siRNAs (18 h incubation), washed, and labeled with Qtracker™ 655 Cell Labeling Kit (ThermoFisher) for 1 h according to the manufacturer's instructions, washed with PBS, and dissociated with 0.05% trypsin. Macrophages were then added to organoids (5000 macrophages added to each organoid) and cultured at 37° C., 5% CO₂, and 1% O₂. Upon combination, organoids are 35 days old, and macrophages are of passage 4-5. Cultures were maintained in 96-well U-bottom plates for 48 hours before assaying.

Immunostaining, RNA extraction and cTNT assays were run after 3 days of culturing.

### Immunofluorescent Staining

Immunofluorescent stainings were performed as described before (Wagner et al. J Mol Cell Cardiol. 2020 January; 138:269-282). After an overnight PFA fixation, the organoids were washed with PBS, then treated with 1% TritonX-100 (in PBS) for 45 minutes. Afterwards, they are incubated with PBS for 10 minutes for washing. 5% horse serum (in PBS) is added to block and incubated for 45 minutes (RT) Primary antibody in 2% horse serum+0.0002% TritonX-100 was added and the samples were incubated at 4° C. overnight. Primary antibodies: Anti-Sarcomeric-actinin (Ab68167, anti-human), Anti-Cleaved Caspase-3 (Ab32042, anti-human). Samples were washed with PBS+0.002% TritonX-100 6×10 min. Secondary antibodies and additional stains were added and incubated for 4 h at RT: AlexaFlour 488 (Invitrogen, A11070), AlexaFlour 555 (Invitrogen, A21425), DAPI, Qtracker labelling 655 (Invitrogen, Q25021MP). Samples were washed by incubating with PBS+0.002% TritonX-100 for 2-3 hours at RT), and mounted on Superfrost Plus Adhesion Slides (ThermoFisher) sing transparent polish to seal the slide sides to the coverslips (Roth). Samples were imaged using SP8 Lightning Leica Confocal Microscope, using a 40× objective (for a total of 800× and 300× magnification with zoom factors and eye lenses). Apoptosis quantification was performed by analyzing the Cleaved-Caspase-3-stained area using FIJI (ImageJ).

cTNT Assay

The cTNT assay was performed using the Human Cardiac Troponin T ELISA@ Kit (ab223860, Abcam) following the manufacturer's instructions.

### Synthetic Lethality Screen

THP-1 monocytes and macrophages were cocultured overnight (18 h) with siRNA. They were then washed with PBS and used for either TET2 knockdown quality control (as described below) or for the drug screening. For the screening, cells were transferred from tissue culture flasks to 384-well plates. Drugs were added to a final concentration of 100 μM and incubated for 48 hours. An Alamar Blue Cell Viability assay (Invitrogen) with a 4 h incubation was performed according to the manufacturer's instructions.

TET2 KD Quality Control (qPCR)

Quality control was performed by extracting RNA from the cells using the RNeasy Mini Kit (Qiagen) following the manufacturer's instructions, and performing qPCR using the RNA to cDNA EcoDry™ Premix (TakaraBio) following the manufacturer's instructions with the following TET2 primers: GTGAGGCTGCAGTGATTGTG (SEQ ID 007), GATTGGTGAGCGTGCCGTAT (SEQ ID 008). The samples were measured using a analyzed using a Applied Biosystems™ PCR system (4376592) and the associated Step One (v2.3) software. Both SYBR™ Green Master Mix (4385612, Applied Biosystems) and TaqMan™ Fast Advanced Master Mix (444557, Applied Biosystems) were used, following manufacturer's instructions.

1. A method for pharmaceutical compound screening comprising the steps
i. providing
a. a cardiac organoid comprising
cardiomyocytes
ii. contacting said cardiac organoid with
b. myeloid cells, or
lymphoid cells, or
hematopoietic stem cells;
thereby yielding a cardiac cell culture model system,
iii. maintaining said cardiac cell culture model system under conditions of cell culture;
wherein a pharmaceutical compound of interest is contacted with
the myeloid cells or the lymphoid cells or the hematopoietic stem cells, before they are contacted with the cardiac organoid; or
the cardiac cell culture model system after step ii,
iv. detecting a read-out of the effect of said pharmaceutical compound on the cardiac cell culture model system,
v. optionally, repeating steps i-iv with a different pharmaceutical compound.

2. The method according to claim 1, wherein the cardiac organoid is a cardiomyocyte monoculture comprising:
cardiomyocytes.

3. The method according to claim 1, wherein the cardiac organoid is a cardiomyocyte biculture comprising:
cardiomyocytes; and
fibroblasts.

4. The method according to claim 1, wherein the cardiac organoid is a triculture comprising:
cardiomyocytes;
endothelial cells; and
fibroblasts.

5. The method according to claim 1, wherein the cardiac organoid is a self-organized cardiac organoid comprising
epicardium;
myocardium;
endocardium; and
cardiac lumen,
wherein said self-organized cardiac organoid comprises:
cardiomyocytes;
endothelial cells;
fibroblasts;
smooth muscle cells;
pericytes;
neurons/sino-atrial node cells.

6. The method according to claim 1, wherein the read-out is detected as one or several effects selected from:
cell viability of the cardiac organoid;
cell viability of myeloid cells or lymphoid cells or hematopoietic stem cells;
cytotoxicity of the lymphoid cells onto the cardiac organoid;
cardiomyocyte cell death;
proliferation of the cells of the cardiac organoid;
proliferation of the myeloid cells or lymphoid cells or hematopoietic stem cells;
proliferation of macrophages;
contractility;
mitochondrial activity
metabolism;
cell infiltration of the myeloid or lymphoid or hematopoietic stem cells into organoid;
inflammation;
reactive oxygen species quantification;
reactive nitrogen species quantification;
fibrosis of the cardiac organoid;
genomics
transcriptomics;
proteomics;
metabolomics.

7. The method according to claim 1, wherein the myeloid cells or lymphoid cells or hematopoietic stem cells comprise a CHIP mutation, wherein the CHIP mutation is a mutation recognized as leading to a clonal hematopoiesis phenotype of the myeloid cells or lymphoid cells or hematopoietic stem cells.

8. The method according to claim 7, wherein the self-organized cardiac organoid does not comprise said CHIP mutation.

9. The method according to claim 7, wherein said CHIP mutation is a mutation in a single gene or a combination of genes selected from TET2, DNMT3A, ASXL1, JAK2, SF3B1, SRSF2, TP53, PPM1D, RUNX1, DDX41, U2AF1, IDH1, IDH2, CBL, KRAS, SMC1A, TERT, SH2B3, CHEK2, ATM/PDGFD, PINT, GFI1B, ABCG1, ABCA1, ABCG4, ABCB6, BCOR, BCORL1, GNB1, and GNAS.

10. The method according to claim 1, wherein the myeloid cells are selected from common myeloid progenitor cells, myeloblasts, megakaryocytes, thrombocytes erythrocytes, mast cells, myeloblasts, basophils, neutrophils, eosinophils, monocytes and macrophages.

11. The method according to claim 1, wherein the lymphoid cells are selected from common lymphoid progenitor cells, small lymphocytes, natural killer cells, small lymphocytes, T-lymphocytes, B-lymphocytes and plasma cells.

12. The method according to claim 1, wherein the myeloid cells or lymphoid cells or hematopoietic stem cells are of different genetic origin than the self-organized cardiac organoid.

13. The method according to claim 1, wherein the pharmaceutical compound is applied to the myeloid cells or the lymphoid cells or hematopoietic stem cells, before they are contacted with the cardiac organoid.

14. The method according to claim 1, wherein the pharmaceutical compound is applied to the cardiac cell culture model system after step ii.

15. The method according to claim 1, wherein the myeloid cells or the lymphoid cells or hematopoietic stem cells are labelled with a dye before step ii.

16. The method according to claim 1, wherein the cardiac cell culture model system is a model for a disease selected from:
clonal hematopoiesis of indeterminate potential (CHIP);
myocarditis;
myocardial inflammation;
viral or bacterial or parasitic infectious disease;
sepsis; and
transplant tissue rejection.

17. The method according to claim 1, wherein step iii of maintaining said cardiac cell culture model system under conditions of cell culture is performed under ˜1% O₂.

18. The method according to claim 1, wherein 3000-8000 cells selected from myeloid cells, lymphoid cells, or hematopoietic stem cells, are added per cardiac organoid, particularly ˜5000 cells are added.

19. (canceled)

20. (canceled)

21. (canceled)

22. A cardiac cell culture model system as described in claim 1.

23. The cardiac cell culture model system according to claim 22, wherein the myeloid cells or lymphoid cells or hematopoietic stem cells comprise a CHIP mutation.
